(19) United States
(12) Patent Application Publication
(10) Pub. No.: US 2010/0131038 A1
(43) Pub. Date: May 27, 2010

(54) STENT ASSEMBLY

(76) Inventors: Zoran Milijasevic, New South Wales (AU); Richard J. Parkinson, New South Wales (AU)

Correspondence Address: Nelson Mullins Riley & Scarborough LLP;IP Department, 100 North Tryon Street, 42nd Floor, Charlotte, NC, 28202-4000, US

(21) Appl. No.: 12/309,450

(22) PCT Filed: Jul. 19, 2007
(86) PCT No.: PCT/AU2007/001004

§ 371 Date: Feb. 4, 2010

Related U.S. Application Data
(60) Provisional application No. 60/820,066, filed on Jul. 21, 2006.

(51) Int. Cl. A61F 2/84; A61F 2/82

(52) U.S. Cl. 623/1.12; 623/1.2

(57) ABSTRACT

A stent assembly (10) comprises a stent component (12). The stent component (12) includes a primary arm (14) defining a first open passage; and at least one secondary arm (16) extending at an angle from the primary arm (14), the at least one secondary arm (16) being integrally formed with the primary arm (14) as a one piece unit and the secondary arm (16) defining a second open passage in communication with the passage of the primary arm (14). An outer sheath (20) surrounds and constrains the stent component (12) in a constricted configuration to facilitate insertion of the stent component 12 into a vascular system of a patient. The outer sheath (20) is removable from the stent component (12) to enable the stent component (12) to adopt its operative configuration when the stent component (12) is at its desired position in the vascular system of the patient.

### CROSS-REFERENCE TO RELATED APPLICATIONS

The present application claims priority from U.S. Provisional Patent Application No. 60/820,066 filed on 21 Jul. 2006, the contents of which are incorporated herein by reference.

### FIELD

This invention relates, generally, to a stent assembly and, more particularly, to a bifurcated stent assembly, to a stent and to a stent delivery system for, and a method of, positioning a stent assembly at a site in a patient's body.

### BACKGROUND

The use of stents to treat lesions caused by the build up of plaque in a patient's vascular system is known. However, a lesion often occurs at a bifurcation in the vascular system. The treatment of such lesions is complex. In the past, two separate stents have been used where one of the stents is passed through an aperture in a side wall of the other stent. Thus, a clinician has to manoeuvre multiple stents which presents additional challenges.

The use of two separate stents can also increase the risk of impeding blood flow, especially to the branch artery. Another problem associated with the use of a separate stent branching from a primary stent is the effect of the stent in the side branch hanging into the main vessel increasing the risk of thrombosis.

Due to the complexity of using two separate stents, some clinicians take the chance of ignoring side branch stenosis. In addition, since lesions rarely occur exactly at the bifurcation but may occur above or below the bifurcation another problem is the effect of shifting the plaque as a result of insertion of the stent or a balloon from one bifurcated vessel into the other.

It is therefore desirable to have a device specifically designed for treating lesions at bifurcations in the vasculature.

### SUMMARY

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect of the invention, there is provided a stent assembly which comprises

a stent component including
a primary arm defining a first open passage; and
at least one secondary arm extending at an angle from the primary arm, the at least one secondary arm being integrally formed with the primary arm as a one piece unit and the secondary arm defining a second open passage in communication with the passage of the primary arm; and

an outer sheath surrounding and constraining the stent component in a constricted configuration to facilitate insertion of the stent component into a vascular system of a patient, the outer sheath being removable from the stent component to enable the stent component to adopt its operative configuration when the stent component is at its desired position in the vascular system of the patient.

In a preferred embodiment, the stent component is bifurcated, being substantially Y-shaped or T-shaped.

Thus, the outer sheath may be shaped to accommodate the bifurcated stent component. The sheath may be tubular having an enlarged distal portion to accommodate the secondary arm of the stent component in a constrained, side-by-side position relative to the primary arm. Instead, the outer sheath may comprise a plurality of interconnected deployment tubes configured to be coincident with the arms of the stent component, at least a portion of the outer sheath being frangible for enabling the outer sheath to be removed from the stent component. In particular, the outer sheath may have a shape complementary to that of the stent component. The frangible portion of the outer sheath may be defined by a zone of weakness extending along at least a part of at least one of the tubes.

One of the outer sheath and the stent component may include a rupture assisting element which facilitates rupturing of the frangible portion of the outer sheath. The rupture assisting element may be arranged in a crook between the primary arm and the secondary arm and may be in the form of a toothed arrangement carried either by the stent component or the outer sheath. In the latter case, the toothed arrangement may be configured to bear against the crook of the stent component to force the parts of the outer sheath on opposite sides of the frangible portion apart upon commencement of withdrawal of the outer sheath from the stent component.

The secondary arm may branch off the primary arm intermediate an inlet opening and an egress opening of the passage of the primary arm.

In an embodiment, the stent component may be a self expanding component made from a shape memory alloy such as Nitinol. In another embodiment, the stent component may be an expansible component which expands under the assistance of a radially outwardly directed force. For example, the stent component may be expanded by inflating a balloon inserted into the component.

Further, the stent component may be polymer coated. In addition, or instead, the stent component may be coated with anti-coagulants, anti-infection surface treatment agents, or other drugs.

According to a second aspect of the invention, there is provided a stent component which comprises

a primary arm defining a first open passage;

at least one secondary arm extending at an angle from the primary arm, the at least one secondary arm defining a second open passage in communication with the passage of the primary arm; and

a rupture assisting element which engages a frangible portion of an outer sheath to assist in rupturing the frangible portion, the rupture assisting element being arranged in a crook between the primary arm and the secondary arm.

The primary arm and the secondary arm may be formed integrally as a one-piece unit.

According to a third aspect of the invention, there is provided a stent component which comprises

a primary arm having a wall portion and defining a first open passage having an inlet opening and an egress opening with at least one intermediate opening being defined in the wall portion; and

a secondary arm associated with the, or each, intermediate opening of the primary arm, the, or each, secondary arm being adjustably attached to the primary arm in register with its associated intermediate opening and the, or each, secondary arm defining a second open passage in communication with the first open passage of the primary arm.

The, or each, secondary arm may be hingedly attached to the primary arm. More particularly, the, or each, secondary arm is hingedly attached to the primary arm by a pair of opposed hinge elements. This allows for independent radial expansion of the primary arm and the, or each, secondary arm.

According to a fourth aspect of the invention, there is provided a stent delivery system for positioning a stent assembly at a site in a patient's body, the system comprising

an introducer;

a stent assembly, as described above, displaceably received within the introducer; and

a guide mechanism extending through the introducer and each arm of the stent assembly for guiding the stent assembly relative to the introducer into position at a desired site in a patient's body.

More particularly, the stent assembly may be displaceably arranged relative to the introducer to be displaced from a first position in which the assembly is received within the introducer and a second position in which the assembly projects from a distal end of the introducer.

The introducer may be a delivery tube having a bore, at least a distal part of the bore being divided into at least two conduits by a septum arrangement. The septum arrangement may be a breakable septum arrangement.

The guide mechanism may comprise an elongate guide element extending through each arm of the stent assembly.

A distal end of each elongate element may carry a trap for entrapping dislodged material. The trap may be a collapsible trap.

At least one of the guide elements may be one of pre-shaped and steerable.

According to a fifth aspect of the invention, there is provided a method of positioning a stent assembly at a site in a patient's body, the method comprising

feeding an introducer containing the stent assembly, as described above, and a guide mechanism to the site at the patient's body;

causing the guide mechanism to extend from a distal end of the introducer so that each of a plurality of elongate guide elements of the guide mechanism is received in a blood vessel at the site;

ejecting the stent assembly from a distal end of the introducer with each arm of the assembly being guided into one of the blood vessels along its associated guide element; and

removing the outer sheath of the assembly to enable each arm of the assembly to be expanded to inhibit restenosis of its associated blood vessel.

The method may include ejecting the stent assembly from the introducer by withdrawing the introducer in a proximal direction, withdrawal of the introducer causing breaking of a septum arrangement arranged at a distal end of the introducer between the arms of the stent assembly.

Further, the method may include removing the outer sheath of the assembly by breaking a frangible portion of the outer sheath. The method may include breaking the frangible portion of the outer sheath by urging the sheath proximally relative to the stent component, a rupture assisting element of the stent component engaging the frangible portion of the outer sheath.

The method may include deploying a trap at a distal end of each guide element to entrap material dislodged from the site. The trap is a collapsible trap and the method may include collapsing the trap after positioning of the stent component to facilitate withdrawal of the guide mechanism.

### BRIEF DESCRIPTION OF DRAWINGS

FIG. 1 shows a three dimensional view of an embodiment of a stent assembly;

FIG. 2 shows a schematic, sectional side view of a part of the stent assembly;

FIG. 3 shows a three dimensional view of a stent component of the assembly;

FIG. 4 shows a schematic end view of a part of an embodiment of a stent delivery system;

FIGS. 5a-5d show various stages in the positioning of the stent component at a site in a patient's body; and

FIG. 6 shows a schematic, sectional side view of a part of another embodiment of a stent component.

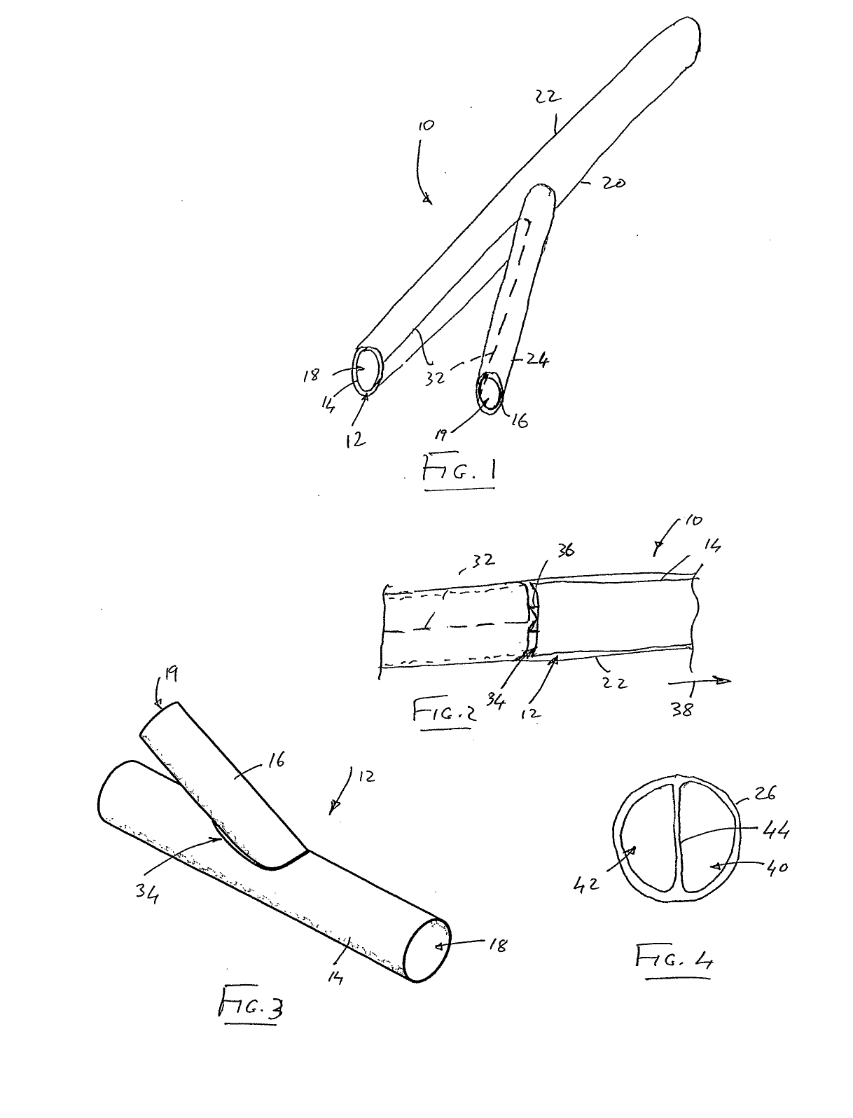

### DETAILED DESCRIPTION OF EXEMPLARY EMBODIMENTS

In the drawings, reference numeral 10 generally designates an embodiment of a stent assembly. The assembly 10 comprises a stent component 12 (FIG. 3). The stent component 12 comprises a primary arm 14 and a secondary arm 16 projecting from the primary arm 14 at a predetermined, acute angle to form a bifurcated stent component 12. The primary arm 14 defines a first, open passage 18. The secondary arm 16 defines a second, open passage 19 opening into, and in communication with, the passage 18 of the primary arm 14.

The assembly 10 further includes an outer sheath 20 which has a shape complementary to that of the stent component 12. Thus, the outer sheath 20 is similarly bifurcated to accommodate the stent component 12. More particularly, the outer sheath 20 has a first, or primary, tube 22 which accommodates the primary arm 14 of the stent component 12 and a second, or secondary, tube 24 which accommodates the secondary arm 16 of the stent component 12.

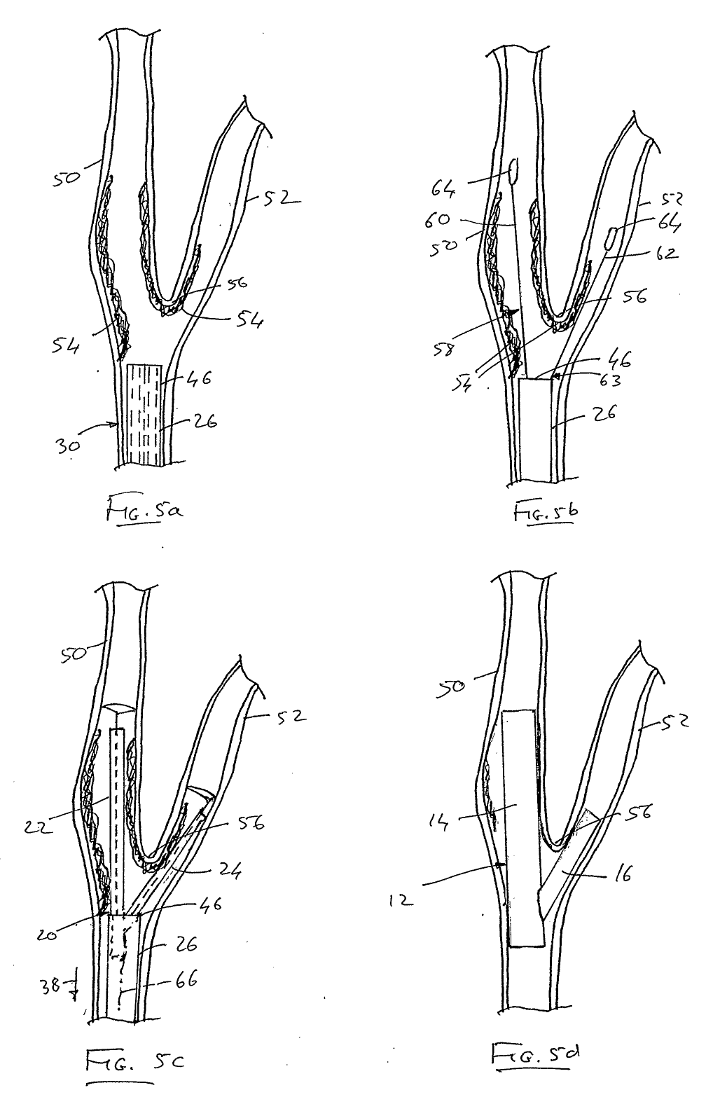

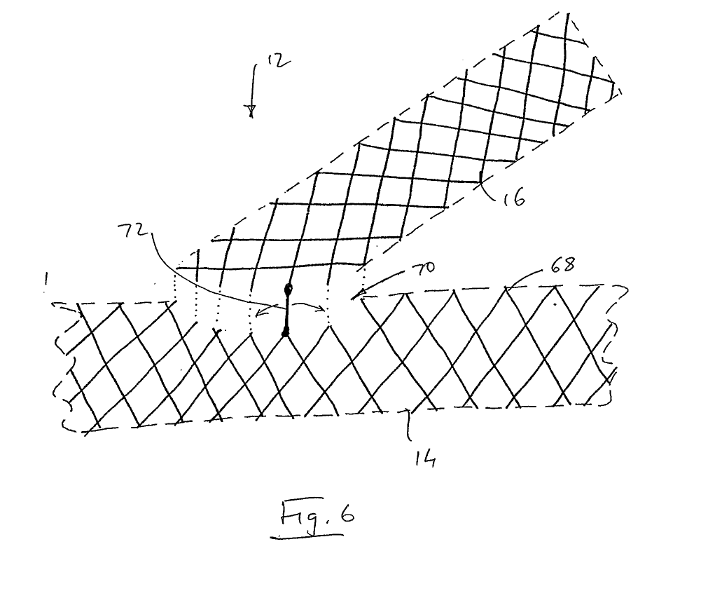

The outer sheath 20 surrounds and contains the stent component 12 in a constricted configuration to facilitate insertion of the stent assembly 10 into an introducer 26 (FIG. 4) of a stent delivery system 30 (FIGS. 5a-5d) and will be described in greater detail below with reference to FIGS. 5a-5d of the drawings.

The outer sheath 20 has a frangible portion 32. More particularly, the frangible portion 32 is a zone of weakness formed in the outer sheath 20 on facing parts of the tubes 22 and 24. In the illustrated embodiment the zone of weakness 32 is a line of perforations. The zone of weakness could instead be an actual break or discontinuity in the wall of the sheath 20 which, upon removal of the introducer 20 from the assembly 10, opens to permit removal of the sheath 20 from the stent component 12.

The junction of the secondary arm 16 with the primary arm 14 of the stent component 12 defines a crook 34. A rupture assisting element 36 (FIG. 2), in the form of a tooth-like formation, is received in the crook 34 of the stent component 12. The rupture assisting element 36 is aligned with the zone of weakness 32 in the outer sheath 20. Consequently, when the outer sheath 20 is urged in the direction of arrow 38 (FIG. 2), the rupture assisting element 36 engages the zone of weakness 32 of the outer sheath 20 causing rupturing of the zone of weakness 32 to facilitate withdrawal of the outer sheath 20 from the stent component 12, as will be described in greater detail below.

In another embodiment, the tooth-like formation is directed oppositely to that illustrated and is carried by the outer sheath 20 to be received in the crook 34 of the stent component 12. The tooth-like formation is configured to bear against the crook 34 of the stent component 12 to force parts of the outer sheath 20 on opposite sides of the zone of weakness 32 apart upon commencement of withdrawal of the outer sheath 20 from the stent component 12.

The stent delivery system 30 comprises the introducer 26 for introducing the stent assembly 10 into the vasculature of the patient undergoing treatment. The introducer 26 is a tubular member. A distal end of the introducer 26 is divided into two conduits 40 and 42 (FIG. 4) by a septum 44. In use, the septum 44 is received between the primary arm 14 and the secondary arm 16 of the stent component 12, while the stent component 12 is enshrouded in the outer sheath 20 to maintain separation of the arms 14 and 16. The septum 44 is of a breakable material and, when the introducer 26 is withdrawn from the vasculature of the patient, the septum 44 breaks to facilitate withdrawal of the introducer 26.

The stent assembly 10 is inserted into the introducer 26 via a proximal end (not shown) of the introducer 26 and is positioned at a distal end 46 of the introducer 26. The introducer 26 is inserted into the vasculature and is steered through the vasculature to the site to be treated. In particular, the assembly 10 is for use in treating a lesion 54 occurring at a bifurcation 48 in the vasculature of the patient such as, for example, at the bifurcation between the external carotid artery 50 and the internal carotid artery 52. Thus, in use, the introducer 26 of the system 30 is steered so that the distal end 46 of the introducer terminates proximally of the bifurcation 56.

A guide mechanism 58, comprising a pair of guide elements, or wires, 60, 62, is extended from the distal end 46 of the introducer 26. The wire 60 of the guide mechanism 58, which feeds into the external carotid artery 50, passes through the passage 18 of the primary arm 14 of the stent component 12. The wire 62, which feeds into the internal carotid artery 52, passes through the passage of the secondary arm 16 of the stent component 12. It will therefore be appreciated that the guide wires 60 and 62 are arranged on opposed sides of the septum 44 of the introducer 26. Further, the guide wire 62 may be pre-shaped with a kink 63 (FIG. 5b) to facilitate insertion of the guide wire 62 into the artery 52.

Each guide wire 60, 62 carries a collapsible trap 64 at its distal end. Prior to deployment of the stent component 12 from within the introducer 26, the traps 64 are opened, as shown in FIG. 5c of the drawings, to entrap material dislodged by releasing or ejecting the stent component 12 from the distal end 46 of the introducer 26.

Once the traps 64 have been opened, the stent assembly 10 is urged out of the distal end 46 of the introducer 26 to the position shown in FIG. 5c of the drawings. When the introducer 26 is withdrawn proximally relative to the stent assembly 10, the tubes 22, 24 of the outer sheath 20 splay outwardly to facilitate insertion of the tubes 22, 24 into their associated arteries 50, 52 respectively. Thus, the tube 22 of the outer sheath 20 is received in the external carotid artery 50 while the tube 24 of the outer sheath 20 is received in the internal carotid artery 52.

A drawstring 66, or the like, is connected to a proximal end of the outer sheath 20. The outer sheath 20 is urged in the direction of arrow 38. The zone of weakness 32′ comes into contact with the rupture assisting element 36 of the stent component 12 causing rupturing of the zone of weakness 32 and facilitating removal of the outer sheath 20 from the stent component 12. Removal of the outer sheath 20 enables the stent component 12 to expand radially to adopt its desired position at the site in the patient's body where restenosis of the site is to be inhibited.

The stent component 12 can be implemented in a number of ways. In one embodiment, the stent component 12, which is of a unitary, one-piece construction, is of a shape memory alloy material such as Nitinol. In this embodiment, the outer sheath 20 constricts the stent component 12 facilitating its insertion into the introducer 26 and its placement at the desired site. Once the outer sheath 20 has been removed, the Nitinol adopts its pre-formed shape which, in this case, is an expanded configuration. This holds the stent component 12 in the desired position at the site.

In another embodiment of the invention, the stent component 12 is of an expansible bio-metal mesh. Once the outer sheath 20 has been removed, a balloon (not shown), in a deflated condition, is inserted into each arm 14, 16 of the stent component 12. The balloon is then inflated to cause a radially outwardly directed force to be exerted on the arms 14 and 16 of the stent component 12 causing the arms 14 and 16 to expand to their desired size.

In both embodiments, instead of the rupture assisting element 36, the outer sheath may be ruptured by inflating the balloon prior to removal of the outer sheath 20. Inflation of the balloon causes rupturing of the outer sheath 20 at the zone of weakness 32 facilitating withdrawal of the outer sheath 20.

FIG. 6 shows a further embodiment of the stent component 12. With reference to the previous drawings, like reference numerals refer to like parts unless otherwise specified. In this embodiment, the secondary arm 16 of the stent component 12 is adjustably attached to the primary arm 14. A radially expansible wall portion 68 of the primary arm 14 defines an intermediate opening 70. The secondary arm 16 is attached to the primary arm 14 so that it is in register with the intermediate opening 70 of the primary arm 14.

It will be appreciated that, in the case of all the embodiments, the stent component 12 could have a plurality of secondary arms 16 associated with the primary arm. In the case of this embodiment, therefore, the wall portion 68 of the primary arm could define more than one intermediate opening 70 with each intermediate opening 70 having a secondary arm 16 associated with it.

The secondary arm 16 is hingedly attached to the primary arm 14. More particularly, the secondary arm 16 is attached to the primary arm 14 by a pair of opposed hinge elements 72 (One of the hinge elements 72 is visible in shown in FIG. 6. The other hinge element is aligned with the visible hinge element). This allows the primary arm 14 and the secondary arm 16 to expand radially independently with respect to each other.

In the case of this embodiment, the guide wires 60 and 62 guide the primary arm 14 and the secondary arm 16, respectively, into their associated arteries 50 and 52. The angle of the secondary arm 16 relative to the primary arm 14 is governed by the angle of the arteries 50 and 52. The angle of the secondary arm 16 relative to the primary arm 14 automatically adjusts to accommodate variations in angles between blood vessels. Due to the presence of the guide wires 60 and 62, there is no need to control or preset the angle of the secondary arm 16 relative to the primary arm 14.

It will be appreciated that, while the stent assembly 10 and the system 30 have been described with reference to their application to the carotid arteries of a patient, the stent assembly 10 and system 30 are able to be used in any other part of the vasculature of a patient's body where bifurcations occur such as, for example, in the coronary arteries.

It is an advantage of the invention that a stent assembly 10 and system 30 are provided which can be used for the treatment of the build up of plaque at bifurcations in a patient's vasculature and can be used without difficulty. In particular, the use of a stent assembly 10 of unitary construction facilitates placement of the stent assembly 10. Due to the use of a one-piece stent component 12, the likelihood of shifting plaque from one vessel to another at the bifurcation is minimised. Also, the use of the one-piece stent component 12 minimises the risk of thrombus occurring at the bifurcation. In addition, a system 30 is provided which facilitates positioning and placement of the stent assembly 10 at the desired location in the patient's vasculature.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A stent assembly which comprises
a stent component including
a primary arm defining a first open passage; and
at least one secondary arm extending at an angle from the primary arm, the at least one secondary arm being integrally formed with the primary arm as a one piece unit and the secondary arm defining a second open passage in communication with the passage of the primary arm; and
an outer sheath surrounding and constraining the stent component in a constricted configuration to facilitate insertion of the stent component into a vascular system of a patient, the outer sheath being removable from the stent component to enable the stent component to adopt its operative configuration when the stent component is at its desired position in the vascular system of the patient.

2. The assembly of claim 1 in which the outer sheath comprises a plurality of interconnected deployment tubes configured to be coincident with the arms of the stent component, at least a portion of the outer sheath being frangible for enabling the outer sheath to be removed from the stent component.

3. The assembly of claim 2 in which the frangible portion of the outer sheath is defined by a zone of weakness extending along at least a part of at least one of the tubes.

4. The assembly of claim 2 in which one of the outer sheath and the stent component includes a rupture assisting element which facilitates rupturing of the frangible portion of the outer sheath.

5. The assembly of claim 4 in which the rupture assisting element is arranged in a crook between the primary arm and the secondary arm.

6. The assembly of claim 1 in which the secondary arm branches off the primary arm intermediate an inlet opening and an egress opening of the passage of the primary arm.

7. The assembly of claim 1 in which the stent component is a self expanding component.

4. The assembly of claim 1 in which the stent component is an expansible component which expands under the assistance of a radially outwardly directed force.

9. A stent component which comprises
a primary arm defining a first open passage;
at least one secondary arm extending at an angle from the primary arm, the at least one secondary arm defining a second open passage in communication with the passage of the primary arm; and
a rupture assisting element which engages a frangible portion of an outer sheath to assist in rupturing the frangible portion, the rupture assisting element being arranged in a crook between the primary arm and the secondary arm.

10. The stent component of claim 9 in which the primary arm and the secondary arm are formed integrally as a one-piece unit.

11. A stent component which comprises
a primary arm having a wall portion and defining a first open passage having an inlet opening and an egress opening with at least one intermediate opening being defined in the wall portion; and
a secondary arm associated with the, or each, intermediate opening of the primary arm, the, or each, secondary arm being adjustably attached to the primary arm in register with its associated intermediate opening and the, or each, secondary arm defining a second open passage in communication with the first open passage of the primary arm.

12. The stent component of claim 11 in which the, or each, secondary arm is hingedly attached to the primary arm.

13. The stent component of claim 12 in which the, or each, secondary arm is hingedly attached to the primary arm by a pair of opposed hinge elements.

14. A stent delivery system for positioning a stent assembly at a site in a patient's body, the system comprising
an introducer;
a stent assembly, as claimed in claim 1, displaceably received within the introducer; and
a guide mechanism extending through the introducer and each arm of the stent assembly for guiding the stent assembly relative to the introducer into position at a desired site in a patient's body.

15. The system of claim 14 in which the introducer is a delivery tube having a bore, at least a distal part of the bore being divided into at least two conduits by a septum arrangement.

16. The system of claim 15 in which the septum arrangement is a breakable septum arrangement.

17. The system of claim 14 in which the guide mechanism comprises an elongate guide element extending through each arm of the stent assembly.

18. The system of claim 17 in which a distal end of each elongate element carries a trap for entrapping dislodged material.

19. The system of claim 18 in which the trap is a collapsible trap.

20. The system of claim 14 in which at least one of the guide elements is at least one of pre-shaped and steerable.

21. A method of positioning a stent assembly at a site in a patient's body, the method comprising
feeding an introducer containing the stent assembly, as claimed in claim 1, and a guide mechanism to the site at the patient's body;
causing the guide mechanism to extend from a distal end of the introducer so that each of a plurality of elongate guide elements of the guide mechanism is received in a blood vessel at the site;
ejecting the stent assembly from a distal end of the introducer with each arm of the assembly being guided into one of the blood vessels along its associated guide element; and
removing the outer sheath of the assembly to enable each arm of the assembly to be expanded to inhibit restenosis of its associated blood vessel.

22. The method of claim 21 which includes ejecting the stent assembly from the introducer by withdrawing the introducer in a proximal direction, withdrawal of the introducer causing breaking of a septum arrangement arranged at a distal end of the introducer between the arms of the stent assembly.

23. The method of claim 21 which includes removing the outer sheath of the assembly by breaking a frangible portion of the outer sheath.

24. The method of claim 23 which includes breaking the frangible portion of the outer sheath by urging the sheath proximally relative to the stent component, a rupture assisting element of the stent component engaging the frangible portion of the outer sheath.

25. The method of claim 21 which includes deploying a trap at a distal end of each guide element to entrap material dislodged from the site.

26. The method of claim 25 in which the trap is a collapsible trap and in which the method includes collapsing the trap after positioning of the stent component to facilitate withdrawal of the guide mechanism.
